(19) United States
(12) Plant Patent Application Publication
(10) Pub. No.: US 2021/0007257 P1
(43) Pub. Date: Jan. 7, 2021

(54) Cannabis Plant Ruby 218

(50) ;

(71) Applicant: MJ Brain Bank, LLC, Denver, CO (US)

(72) Inventor: Kevin Sullivan, Broomfield, CO (US)

(21) Appl. No.: 16/501,911

(22) Filed: Jul. 3, 2019

(51) Int. Cl. A01H 6/28

(52) U.S. Cl. PLT/258

(57) ABSTRACT

‘Ruby 218’ is new Cannabis plant variety that has useful characteristics such as high potency and sweet aroma.

Genus and species: The Cannabis variety of this invention is botanically identified as Cannabis sativa.

Variety denomination: The variety denomination is ‘Ruby 218’.

### BACKGROUND OF THE INVENTION

The invention refers to a new plant variety of Cannabis (Cannabis sativa) named ‘Ruby 218’. The new Cannabis variety is product of a planned and controlled breeding program for new Cannabis varieties. It was first asexually reproduced in Denver, Colo., and it has since also been asexually reproduced in Trinidad, Colo. The objective of the breeding program is to select new Cannabis varieties with a combination of desired vigor, potency, aroma, bud structure/form, yield, and tolerance or resistance to diseases.

The new Cannabis variety is a product of a planned breeding program intended to combine some of the desirable characteristics of two existing varieties: Rubicon and Pure Afghan. The variety arose from a cross between the parental varieties; using Pure Afghan as the pollen donor. The parental cross was made in August 2014. Single plants were selected and reproduced by cuttings using traditional horticultural plugs, trays and domes. One of the reproduced plants showed pronounced vigor and was selected as and named ‘Ruby 218’. This plant was selected and transferred to the production in April 2015. The plant has been asexually reproduced by cuttings since that time. The distinctive characteristics of the variety have been stable since that time through successive generations.

For asexual reproduction, axillary branches of the plant were excised from the mother plant. The cuttings were treated with indole 3 butyric acid; the cuttings were allowed to root over a period of three weeks at a temperature of 77 degrees F., relative humidity of 60%-80% and constant illumination at an intensity of 100 PPFD.

### BRIEF SUMMARY OF THE INVENTION

The following traits have been repeatedly observed and are determined to be unique characteristics of ‘Ruby 218’ which in combination distinguish this Cannabis plant as a new and distinct variety, as compared to the parents, the closet varieties:

1. The new variety ‘Ruby 218’ has higher CBD content as compared to the parents, an indication of a higher potency,

2. The new variety has a sweet grass aroma as compared to the citrus/fruity or herbal/spicy aroma of the parents.

3. The new variety produces higher number of flowers per plant and the flowers of the new variety have larger diameter as compared to the parents, an indication of a higher yield.

4. The new variety has an above average yield per plant due to its vigor and inflorescence structure which allows for several large and heavy clusters to form on each plant. Flower may remain salable, marketable and usable for up to 1 year after harvest with minimal degradation or impact on use.

The traits of the new variety was tested at 2600 West Barberry Pl. Denver, Colo., U.S.A.

### BRIEF DESCRIPTION OF THE PHOTOGRAPHS

The accompanying photographs illustrate the appearance of the new Cannabis variety when the plants were at 63 days after flower initiation. The colors in the photographs are depicted as nearly true as is reasonably possible to obtain in colored reproductions of this type. Colors as shown in the photographs may differ slightly from the color values cited in the detailed botanical description.

FIG. 1 shows a branch of a plant of the new variety.

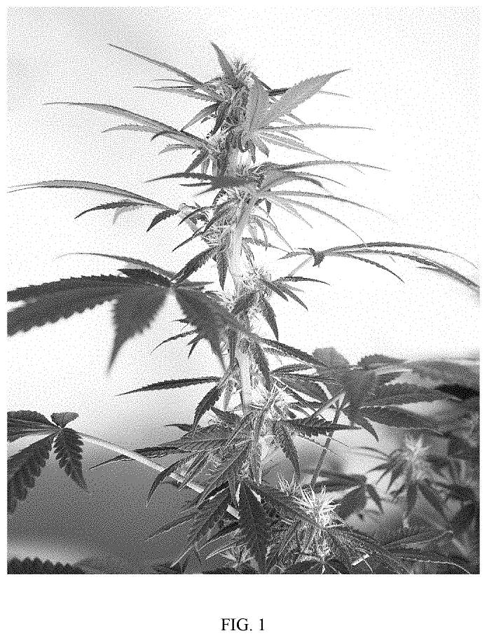

FIG. 2 shows the side view of a plant of the new variety.

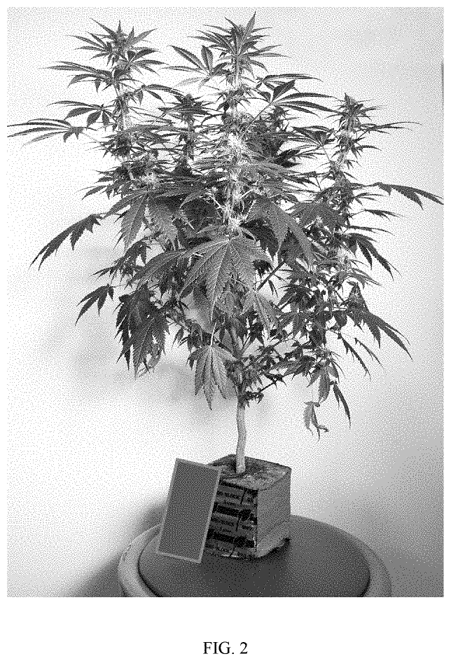

FIG. 3 shows the top view of a flowering plant of the new variety in close-up.

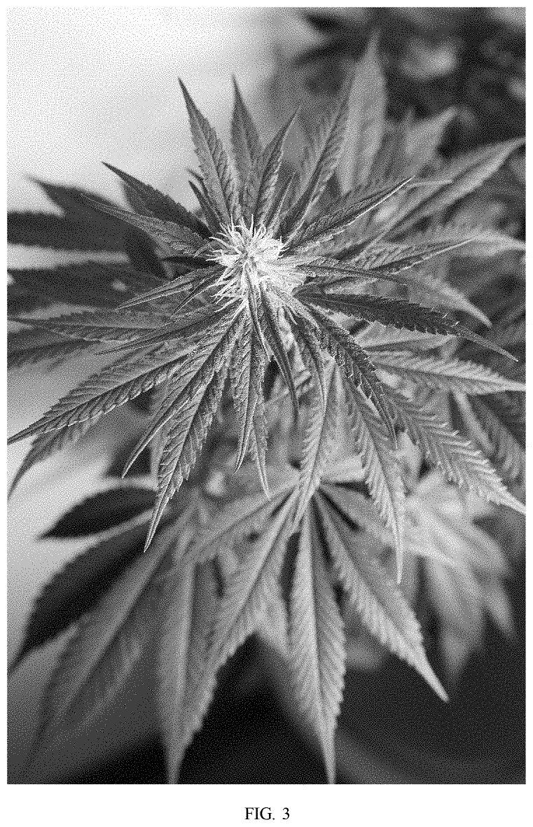

FIG. 4 shows a dried bud of a plant of the new variety.

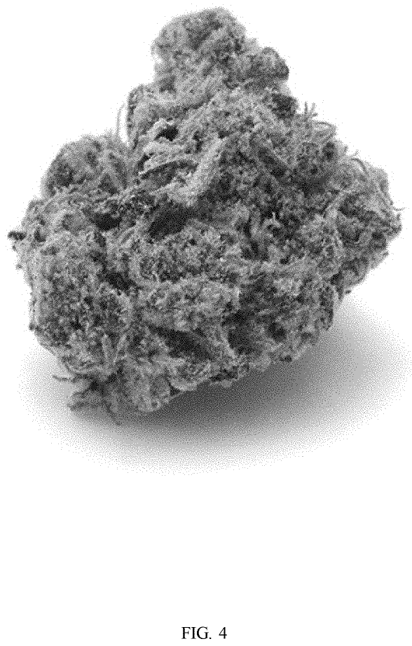

FIG. 5 shows flowers of a plant of the new variety.

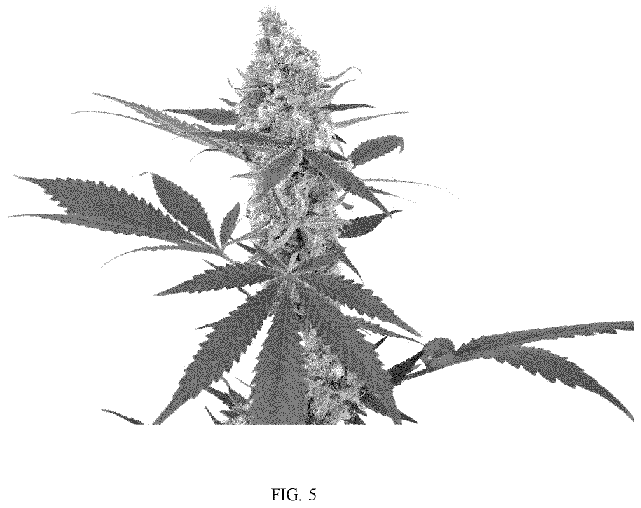

### DETAILED BOTANICAL DESCRIPTION

The following description is based on observations made in February of 2016 on plants produced according to an ordinary growth cycle. However, the new Cannabis variety ‘Ruby 218’ has not been observed under all possible environmental conditions. The phenotype of the new variety may vary with variations in environmental conditions such as temperature, light intensity and day length without any change in the genotype of the plant.

The photographs and description are taken from plants grown at the 2600 West Barberry Pl. in Denver, Colo., U.S.A. Following rooting, the cuttings were transplanted into Hydroton, and were grown for approximately 6 weeks or until roots emerged. The plants were then allowed to grow additional 9 weeks. During growth, all plants were fed and watered with nutrients from the Botanicare Kind line. Carbon dioxide concentration was measured and supplemented as required, between 900-1500 ppm. Vegetative growth was at a temperature of 78 degrees F., and relative humidity of 45% with constant illumination of 600 PPFD provided by 600W Metal Halide grow lights. After adequate vegetative growth had been attained, flowering was induced by changing the photoperiod to 12 hours of light and 12 hours of darkness.

Plants were observed on day 63 after the flower induction and the botanical description thereof is provided below. Characteristics described represent those which are typical for the new variety. Measurements and numerical values represent averages for 12 typical plants and plant parts. The actual measurements of any individual plant of plant parts, or any group of plants of plant parts, may vary from the stated averages.

### PLANT DESCRIPTION

TABLE I
General

| | | Parental | | Check Variety |
|---|---|---|---|---|
| | | variety | Parental variety | (Cannabis |
| Characteristic | New Variety | (Female plant) | (Pollen donor) | sativa) |
| Plant life forms | Herbaceous | Herbaceous | Herbaceous Plant | Herbaceous |
| | Plant | Plant | | Plant |
| Plant growth habit | Upright, tap- | Upright, tap- | Upright, tap- | Upright, tap- |
| | rooted annual | rooted annual | rooted annual | rooted annual |
| | plant | plant | plant | plant |
| Plant origin | F1 hybrid | F1 hybrid | Landrace variety | F1 hybrid |
| | between two | between two | from Mazar I | between two |
| | Cannabis | Cannabis | Sharif | Cannabis |
| | Subspecies | Subspecies | | Subspecies |
| | (Rubicon × | (Afghani × | | (Lambs Bread × |
| | Pure Afghan) | Shunk #1) | | Sour Diesel) |
| Plant propagation | Asexual | Asexual | Asexual | Asexual |
| Propagation ease | Easy¹ | Easy | Moderate² | Moderate |
| Height | Variable; 1 | Variable; 1 | Variable; 1 meter | Variable; 1 |
| (Unit: m or cm) | meter | meter | | meter |
| Width | Variable; | Variable; 0.75 | Variable; 0.75 | Variable; 0.75 |
| (Unit: m or cm) | 0.75 meters | meters | meters | meters |
| Plant vigor | High | High | Medium | High |
| Time to Harvest | 18-20 weeks | 18-20 weeks | 18-20 weeks | 18-20 weeks |
| (Seed to Harvest) | | | | |
| Resistance to pests or | No | No | No | No |
| diseases? | | | | |
| Is this plant a | No | No | No | No |
| Genetically Modified | | | | |
| Organism? | | | | |

¹: Easy refers to plants that don't require any special products, chemicals or hormones for rooting. Roots will also show up within 10-14 days.
²: Moderate refers to plants that may take 14-21 days with an increase in rooting after additional hormone or nutrient has been introduced.

Additional general characteristics:

The variety is susceptible to powdery mildew and spider mites.

TABLE II
Leaf/Foliage

| | | Parental | | |
|---|---|---|---|---|
| | | variety | | |
| | | (Female | Parental variety | |
| Characteristic | New Variety | plant) | (Pollen donor) | Check Variety |
| Leaf arrangement | Alternate | Alternate | Alternate | Alternate |
| (Phyllotaxy) | | | | |
| Leaf shape | Palmately | Palmately | Palmately | Palmately |
| | compound | compound | compound | compound |
| Leaf structure | Palmately | Palmately | Palmately | Palmately |
| | compound | compound | compound | compound |
| Leaf margins | Coarsely | Coarsely | Coarsely serrated | Coarsely |
| | serrated | serrated | pointing towards | serrated |
| | pointing | pointing | tip of leaflet | pointing |
| | towards tip of | towards tip | | towards tip of |
| | leaflet | of leaflet | | leaflet |
| Leaf hairs | Absence | Absence | Absence | Absence |
| (Presence or absence) | | | | |
| Leaf length with | 20 cm | 16 cm | 18 cm | 18 |
| petiole at maturity | | | | |
| (Unit: cm) | | | | |
| Petiole length at | 10 cm | 9 cm | 10 cm | 12 |
| maturity | | | | |
| (Unit: cm) | | | | |
| Stipule length at | 1 cm | 1 cm | 1 cm | 1 cm |
| maturity | | | | |
| (Unit: cm or mm) | | | | |
| Stipule shape | Long, bulbous | Long, | Long, bulbous | Long, bulbous |
| | bases with | bulbous | bases with | bases with |
| | acuminate | bases with | acuminate | acuminate |
| | (tapering | acuminate | (tapering concave | (tapering |
| | concave to | (tapering | to apex) shape | concave to |
| | apex) shape | concave to | | apex) shape |
| | | apex) shape | | |
| No. of leaflets | 5-9 | 5-9 | 5-9 | 5-9 |
| (a range is OK) | | | | |
| Middle largest | 9 | 9 | 8 | 8 |
| (longest) leaflet length | | | | |
| (Unit: cm) | | | | |
| Middle largest | 2 | 1.5 | 2 | 2 |
| (longest) leaflet width | | | | |
| (Unit: cm) | | | | |
| Middle largest | 9:2 | 6:1 | 4:1 | 4:1 |
| (longest) leaflet | | | | |
| length/width ratio | | | | |
| No. teeth of middle | 14 | 14 | 13 | 13 |
| leaflet (average) | | | | |
| Leaf (upper side) color | 141C | 141C | 141C | 141C |
| (RHS³ No.) | | | | |
| Leaf (lower side) color | 143A | 143A | 143A | 143A |
| (RHS No.) | | | | |
| Leaf glossiness | Medium | Medium | Medium | Medium |
| Vein/midrib shape | Even, not | Even, not | Even, not | Even, not |
| (general description) | alternate. | alternate. | alternate. Slightly | alternate. |
| | Slightly points | Slightly | points towards tip | Slightly points |
| | towards tip just | points | just like the | towards tip just |
| | like the | towards tip | serrations | like the |
| | serrations | just like the | | serrations |
| | | serrations | | |
| Vein/midrib color | Light green | Light green | Light green | Light green |
| | (RHS 140B) | (RHS 140B) | (RHS 140B) | (RHS 140B) |
| Aroma | Sweet grass | Citrus/Fruity | Herbal/Spicy | Gas/Fuel, |
| (general description) | | | | Earthy |

³As used in this disclosure, RHS stands for Royal Horticultural Society Colour Chart, 5^{th} Edition, which can be found at: rhscf.orgfree.com

Additional Leaf/Foliage-related Characteristics of the new variety

Petiole and stipule surface texture and color designation: Smooth petiole and stipule; RHS color 140B

Anthocyanin coloration on petiole: Very weak

TABLE III
Stem

| | | Parental | | |
|---|---|---|---|---|
| | | variety | Parental | |
| | | (Female | variety | |
| Characteristic | New Variety | plant) | (Pollen donor) | Check Variety |
| Stem shape | hollow, large, | hollow, | hollow, large, | hollow, large, |
| (general description) | rugose, | large, rugose, | rugose, | rugose, punctuate, |
| | punctuate, | punctuate, | punctuate, | minutely |
| | minutely | minutely | minutely | glandular, ribbed |
| | glandular, ribbed | glandular, | glandular, | with ribs running |
| | with ribs running | ribbed with | ribbed with | parallel to stem |
| | parallel to stem | ribs running | ribs running | |
| | | parallel to | parallel to stem | |
| | | stem | | |
| Stem diameter at | 1.25 cm | 1 cm | 1.5 cm | 1 cm |
| base | | | | |
| (Unit: cm) | | | | |
| Stem color | 144D | 144D | 144D | 144D |
| (RHS No.) | | | | |

Additional stem characteristics of the new variety:

Depth of main stem grooves: Shallow

Length of internode: Medium

TABLE IV
Inflorescence (Female /Pistillate Flowers)

| | New Variety | Parental variety | Parental variety | |
|---|---|---|---|---|
| Characteristic | (Female plant) | (Female plant) | (Pollen donor) | Check Variety |
| Flowering | Dioecious. Forms | Dioecious. Forms | Dioecious. Small | Dioecious. |
| (blooming) | a tall | a tall | inflorescences, | |
| habit (General | interconnected | interconnected | tightly packed | |
| description) | cola or terminal | cola or terminal | against stem and | |
| | bud | bud | separated all | |
| | | | along plants | |
| | | | branches. | |
| Proportion of | 50% | N/A | N/A | N/A |
| female plants | | | | |
| (50% or less?) | | | | |
| Inflorescence | Above | Above | Above | Above |
| position | | | | |
| Flower | Overlapping⁵ | Overlapping | Overlapping | Overlapping |
| arrangement | | | | |
| Number of | 250 | 200 | 175 | 200 |
| flowers per | | | | |
| plant | | | | |
| Flower shape | Each flower has a | Each flower has a | Each flower has a | Each flower has a |
| | small green bract | small green bract | small green bract | small green bract |
| | enclosing the | enclosing the | enclosing the | enclosing the |
| | ovary with two | ovary with two | ovary with two | ovary with two |
| | long, slender | long, slender | long, slender | long, slender |
| | stigmas | stigmas | stigmas | stigmas |
| | projecting well | projecting well | projecting well | projecting well |
| | above the bract. | above the bract. | above the bract. | above the bract. |
| | And the-flower | And the flower | And the flower | And the flower |
| | looks urceolate | looks urceolate | looks urceolate | looks urceolate |
| | (urn-shaped). | (urn-shaped). | (urn-shaped). | (urn-shaped). |
| Flower | 3 mm | 3 mm | 3 mm | 3 mm |
| (individual | | | | |
| pistillate) | | | | |
| length | | | | |
| (Unit: mm) | | | | |
| Flower | 2.5 cm | 2 cm | 1.5 cm | 2 cm |
| diameter | | | | |
| (Unit: cm) | | | | |
| (compound | | | | |
| cyme) | | | | |
| Corolla | Round | Round | Round | Round |
| (general | | | | |
| description) | | | | |
| Corolla Color | 142C | 142C | 142C | 142C |
| (RHS No.) | | | | |
| Bract shape | Round at the base | Round at the base | Round at the base | Round at the base |
| (general | but tapered | but tapered | but tapered | but tapered |
| description) | towards the tip | towards the tip | towards the tip | towards the tip |
| Bract color | 142C | 142C | 142C | 142C |
| (RHS No.) | | | | |
| Calyx shape | Round | Round | Round | Round |
| (general | | | | |
| description) | | | | |
| Calyx color | 142C | 142C | 142C | 142C |
| (RHS No.) | | | | |
| Stigma shape | Curved and | Curved and | Curved and | Curved and |
| (general | wavy, thin | wavy, thin | wavy, thin | wavy, thin |
| description) | | | | |
| Stigma length | 0.6 cm | 0.6 cm | 0.6 cm | 0.6 cm |
| (Unit: mm) | | | | |
| Stigma color | closely | closely | closely | closely |
| (RHS No.) | resembling an off | resembling an off | resembling an off | resembling an off |
| | white or | white or | white or | white or |
| | NN155D | NN155D | NN155D | NN155D |
| Trichome | Glandular, thus it | Glandular, thus it | Glandular, thus it | Glandular, thus it |
| shape | has a stalk that | has a stalk that | has a stalk that | has a stalk that |
| (general | supports | supports | supports | supports |
| description) | secretory head | secretory head | secretory head | secretory head |
| Trichome | Mature cap = | Mature cap = | Mature cap = | Mature cap = |
| color (RHS | N172C | N172C | N172C | N172C |
| No.) | Stalk = | Stalk = | Stalk = | Stalk = |
| | Clear/Translucent | Clear/Translucent | Clear/Translucent | Clear/Translucent |
| | closely | closely | closely | closely |
| | resembling an off | resembling an off | resembling an off | resembling an off |
| | white or | white or | white or | white or |
| | NN155D | NN155D | NN155D | NN155D |
| Terminal bud | tall and | Somewhat tall | Small, rounded | Round but with |
| shape | interconnected | and | and slightly flat | textured |
| (general | cola or terminal | interconnected | on top. | projections (fox |
| description) | bud. Lance | cola or terminal | | tails). Spikey |
| | | bud. Conical | | |
| Terminal bud | 142C | 142C | 142C | 142C |
| color (RHS | | | | |
| No.) | | | | |
| Pedicel | absence | absence | absence | absence |
| (Presence or | | | | |
| absence) | | | | |
| Staminate | N/A | N/A | Round sacks of | N/A |
| shape | . | | modular | |
| | | | composition like | |
| | | | the hand of a | |
| | | | banana but | |
| | | | originating from | |
| | | | a set of stems | |
| | | | similar to grapes | |
| Pollen | N/A | N/A | Typical yellow | N/A |
| description | | | pollen with | |
| | | | musty smell | |
| Seed | Starts off white | Starts off white | N/A | Starts off white |
| description | and turns to | and turns to | | and turns to |
| | Brown with black | Brown with black | | Brown with black |
| | or dark brown | or dark brown | | or dark brown |
| | stripes | stripes | | stripes |
| Petal | absence | absence | absence | absence |
| description | | | | |
| THC and/or | >1% THC | 9-12% THC | N/A (male) | 20-24% |
| CBD content⁶ | 16-20% CBD | 9-12% CBD | | |

⁵Overlapping, when referring to flower arrangement, means that the leaves from each shoot overlap with each other and obscure the flowers.
⁶THC and/or CBD content were determined using High Performance Liquid Chromatography (“HPLC”) with Diode-Array Detection (“DAD”).

Additional inflorescence-related characteristics of the new variety:

Proportion of hermaphrodite and male plants: Medium

Average size of terminal bud: 8-10 inches

Corolla, bract and calyx surface textures and average sizes: 4 mm bracts once fully developed; texture is sticky and slightly rough due to trichomes and the floral density.

Immature glandular trichomes color designation on the flower: transparent and clear, most similar to 3D.

Types of trichomes: Non-glandular and capitate-stalked. Non-glandular covers the new growth and some rachis until the plant is in flower. Once in flower they will continue but the capitate stalked trichomes being to appear. They can be found on all parts of the flower as well as the petiole and again on the rachis. All are transparent and clear and until the capitate stalked mature into a color close to 17A, they will be closer to 3D or colorless.

Seed average size and color designation: Average size is 4 mm in diameter. Color equals 199A.

Intensity of seed striping: Strong

TABLE V
Other Characteristics and Culture

| | | Parental | | |
|---|---|---|---|---|
| | | variety | Parental | |
| | New Variety | (Female | variety | |
| Characteristic | (Female plant) | plant) | (Pollen donor) | Check Variety |
| Time period and | Blooms in 9-11 | Blooms in 9- | Blooms in 9-11 | Blooms in 9-11 |
| condition of | weeks after | 11 weeks | weeks after | weeks after |
| flowering/blooming | cutting-and- | after cutting- | cutting-and- | cutting-and- |
| | rooting when | and-rooting | rooting when | rooting when |
| | more than 12 | when more | more than 12 | more than 12 |
| | hours light is | than 12 | hours light is | hours light is |
| | applied to induce | hours light is | applied to | applied to induce |
| | flowering | applied to | induce | flowering |
| | | induce | flowering | |
| | | flowering | | |
| Hardiness of plant | High⁷ | High | Medium | Medium |
| Breaking action⁴ | High | High | Medium | Medium |
| Rooting rate after | 9 days | 9 days | 9 days | 9 days |
| cutting/cloning | | | | |
| (under certain or | | | | |
| specific condition) | | | | |

⁷A high hardiness refers to drought resistant; low nutrient requirement/demand; wind resistant due to strong branching and turgidity, along with above average rooting ability (anchoring).
⁴refers to how strong the stem is against the breakage such as fibrous, strong, flexible, resistant to breakage, etc.

1. A new and distinct variety of Cannabis plant having the characteristics substantially as described and illustrated herein.
